(19) United States
(12) Patent Application Publication
(10) Pub. No.: US 2013/0236934 A1
(43) Pub. Date: Sep. 12, 2013

(54) RECOMBINANT BACTERIUM FOR L-HOMOSERINE PRODUCTION

(71) Applicant: NOVUS INTERNATIONAL INC., St. Charles, MO (US)

(72) Inventors: Ming Kang, Lincoln, NE (US); Muralidhar Tata, Lincoln, NE (US); Sridhar Vakalapudi, Lincoln, NE (US); Patrick McLaughlin, Lincoln, NE (US); Yingjie Ma, Lincoln, NE (US); Aditya Mahajan, Lincoln, NE (US); David Wickard, Lincoln, NE (US); Stephen Lorbert, St. Charles, MO (US); Friedhelm Brinkhaus, De Moines, IA (US); James C. Peterson, St. Charles, MO (US)

(73) Assignee: NOVUS INTERNATIONAL INC., St. Charles, MO (US)

(21) Appl. No.: 13/790,589

(22) Filed: Mar. 8, 2013

Related U.S. Application Data
(60) Provisional application No. 61/608,325, filed on Mar. 8, 2012.

(51) Int. Cl. C12P 13/06

(52) U.S. Cl. CPC C12P 13/06
USPC 435/116; 435/252.33

(57) ABSTRACT

The present invention encompasses a recombinant bacterium that is capable of producing L-homoserine and methods of using the bacterium for producing L-homoserine.

### CROSS REFERENCE TO RELATED APPLICATIONS

This application claims the priority of U.S. provisional application No. 61/608,325, filed Mar. 8, 2012, which is hereby incorporated by reference in its entirety.

### FIELD OF THE INVENTION

The present invention relates to recombinant bacterium suitable for L-homoserine production, and methods of use thereof.

### BACKGROUND OF THE INVENTION

Homoserine is a precursor and/or intermediate for the biosynthesis of several essential amino acids such as threonine, isoleucine, and methionine. Efforts have been made to produce homoserine in E. coli by expressing enzymes from the threonine synthesis pathway. However, these attempts used expression systems with strong non-native promoters to control the expression of the enzymes from the threonine synthesis pathway, and did not enhance the yield of L-homoserine considerably. Therefore, there is a need for a more efficient method of producing L-homoserine in recombinant bacteria.

### SUMMARY OF THE INVENTION

One aspect of the invention encompasses a recombinant bacterium for producing L-homoserine. A recombinant bacterium comprises one or more exogenous nucleic acids encoding a polypeptide with aspartokinase activity, one or more exogenous nucleic acids encoding a polypeptide with homoserine dehydrogenase activity, one or more exogenous nucleic acids encoding a polypeptide with phosphoenolpyruvate carboxylase activity, and one or more exogenous nucleic acids encoding a polypeptide with homoserine transport activity, and attenuated expression of the genomic nucleic acid encoding a polypeptide with homoserine kinase activity. The one or more of the exogenous nucleic acids are operably linked to a native promoter.

Another aspect of the invention encompasses a method of producing L-homoserine. The method comprises cultivating a recombinant bacterium of the invention in a culture medium and collecting the L-homoserine from the medium.

Other aspects and iterations of the invention are described more thoroughly below.

### BRIEF DESCRIPTION OF THE FIGURES

FIG. 1 depicts SDS-PAGE gels showing AKI-HdHI expression from the thrA* nucleic acid under control of the Ptac promoter and induced by IPTG. (A) AKI-HdHI protein in supernatant and pellet fractions of CGSC 8333 bacteria comprising the pNI2 plasmid after induction with increasing concentrations of IPTG. (B) AKI-HdHI protein in supernatant fractions of CGSC 8333 or MG1655 bacteria comprising the pNI2 plasmid, after induction with IPTG for various durations.

FIG. 2 depicts agarose gels showing stability of plasmids comprising the Ptac promoter with or without the lacI repressor gene during fermentation. (A) Restriction digest of plasmid DNA extracted from E. coli K-12 strain ATCC98082 and CGSC8333 transformed with pNI1. (B) Restriction digest of plasmid DNA extracted from E. coli K-12 strain ATCC98082 and CGSC8333 transformed with pNI1.6.

### DETAILED DESCRIPTION OF THE INVENTION

The present invention provides a recombinant bacterium capable of producing L-homoserine. In particular, the present invention provides a recombinant bacterium capable of producing L-homoserine and secreting L-homoserine into a medium when the bacterium is cultured in the medium. The invention also provides a method of producing L-homoserine by cultivating the bacterium in a culture medium to produce and secrete L-homoserine into the medium, and collecting the L-homoserine from the medium.

### I. Recombinant Bacterium

One aspect of the invention encompasses a recombinant bacterium for producing L-homoserine. A recombinant bacterium of the invention typically belongs to the Enterobaceteriaceae. The Enterobacteria family comprises species from the following genera: Alterococcus, Aquamonas, Aranicola, Arsenophonus, Brenneria, Budvicia, Buttiauxella, Candidatus Phlomobacter, Cedecea, Citrobacter, Edwardsiella, Enterobacter, Erwinia, Escherichia, Ewingella, Hafnia, Klebsiella, Kluyvera, Leclercia, Leminorella, Moellerella, Morganella, Obesumbacterium, Pantoea, Pectobacterium, Photorhabdus, Plesiomonas, Pragia, Proteus, Providencia, Rahnella, Raoultella, Salmonella, Samsonia, Serratia, Shigella, Sodalis, Tatumella, Trabulsiella, Wigglesworthia, Xenorhabdus, Yersinia, Yokenella. In certain embodiments, a recombinant bacterium is typically of the Escherichia genus. In exemplary embodiments, a recombinant bacterium may be Escherichia coli. In a particularly exemplary embodiment, a recombinant bacterium is an E. coli strain comprising attenuated expression of the genomic thrB nucleic acid encoding a polypeptide with homoserine kinase activity as described below.

A recombinant bacterium of the invention may express one or more nucleic acids, or comprise one or more mutations for producing L-homoserine as detailed below. In particular, a bacterium capable of producing L-homoserine may express one or more nucleic acids, or comprise one or more mutations to enhance synthesis, accumulation and secretion of L-homoserine into the medium.

Methods of expressing one or more nucleic acids are known in the art. In general, a bacterium may be transformed with one or more vectors comprising nucleic acid constructs for producing L-homoserine. Methods of transformation are well known in the art, and may include electroporation, natural transformation, and chemical transformation (e.g. calcium chloride, rubidium chloride, etc.). Methods of introducing a mutation into a bacterium are known in the art and may include deletion mutations and insertion-deletion mutations.

### (a) Nucleic Acids

A recombinant bacterium capable of producing L-homoserine may comprise one or more exogenous nucleic acids, or comprise one or more mutations to enhance synthesis and accumulation of L-homoserine. L-homoserine is an intermediate amino acid in the metabolic pathway depicted in the diagram below that produces L-lysine, L-methionine, L-isoleucine, glycine and L-threonine. As used herein, “exogenous nucleic acid” refers to a nucleic acid sequence that is not typically present in the wild-type genome of the particular microorganism.

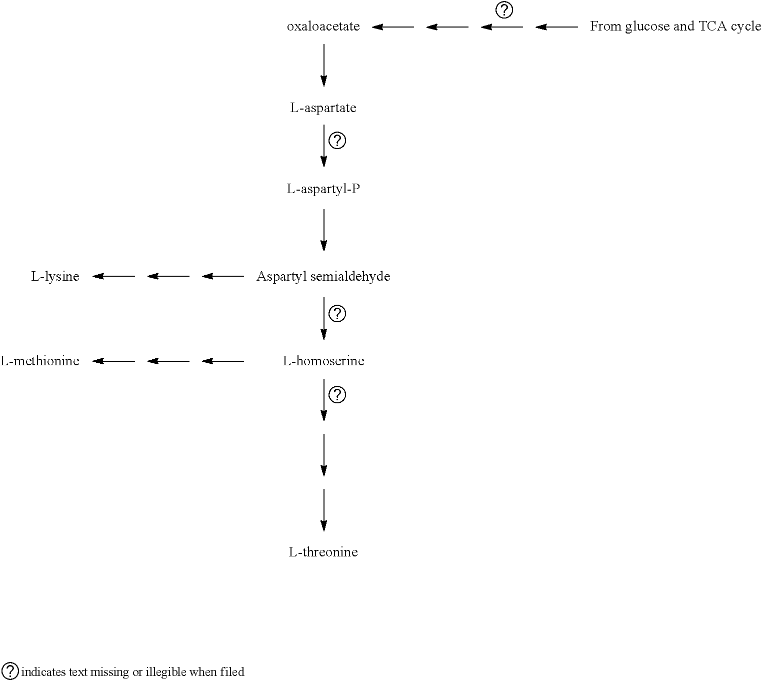

In some embodiments, a recombinant bacterium for producing L-homoserine may comprise one or more exogenous nucleic acids encoding one or more polypeptides. In some embodiments, a recombinant bacterium may comprise one, two, three, four or more exogenous nucleic acids. In preferred embodiments, a recombinant bacterium may comprise at least three exogenous nucleic acids. In other preferred embodiments, a recombinant bacterium may comprise at least two exogenous nucleic acids. In an exemplary embodiment, a recombinant bacterium for producing L-homoserine may comprise three nucleic acids from a plasmid vector as described below. In another exemplary embodiment, a recombinant bacterium for producing L-homoserine may comprise two nucleic acids from a plasmid vector as described below.

A recombinant bacterium for producing L-homoserine may comprise one or more exogenous nucleic acids encoding one or more polypeptides with enzyme activities for synthesizing intermediates in the L-threonine synthesis pathway leading to the synthesis of L-homoserine. Non-limiting examples of enzymes that synthesize intermediates in the L-threonine synthesis pathway may include aspartokinase, aspartyl semialdehyde dehydrogenase, and homoserine dehydrogenase. In some embodiments, a recombinant bacterium may comprise exogenous nucleic acids encoding one or more polypeptides with aspartokinase, aspartyl semialdehyde dehydrogenase, and homoserine dehydrogenase activities. In exemplary embodiments, a recombinant bacterium may comprise exogenous nucleic acids encoding one or more polypeptides with aspartokinase and homoserine dehydrogenase activities. In a particularly exemplary embodiment, a recombinant bacterium for producing L-homoserine may comprise an exogenous E. coli thrA nucleic acid sequence encoding a polypeptide with dual aspartokinase and homoserine dehydrogenase activities.

The L-threonine synthesis pathway is regulated, in part, by feedback inhibition. For instance, the activity of homoserine kinase, homoserine dehydrogenase and aspartokinase are inhibited by the accumulation of L-threonine or L-homoserine. In some embodiments, a recombinant bacterium may comprise exogenous nucleic acids encoding mutant versions of enzymes in the L-threonine synthesis pathway that are free of feedback inhibition. In preferred embodiments, a recombinant bacterium may comprise a mutant version of an exogenous E. coli thrA nucleic acid sequence encoding a polypeptide with homoserine dehydrogenase and aspartokinase activities that is free of feedback inhibition. Non-limiting examples of thrA nucleic acid sequences encoding a polypeptide with homoserine dehydrogenase and aspartokinase activities that are free of feedback inhibition include the thrA* mutation from the E. coli strain ATCC21277, thrA₁I, thrA₂I, and carboxy terminal deletions to thrA. In an exemplary embodiment, a recombinant bacterium may comprise the mutant thrA* exogenous nucleic acid sequence from the E. coli strain ATCC21277 encoding a polypeptide with homoserine dehydrogenase and aspartokinase activities that are free of feedback inhibition.

In other embodiments, a recombinant bacterium may comprise attenuated expression of polypeptides with enzyme activities that use and deplete L-homoserine in a bacterium, or enzyme activities that use and deplete intermediates that lead to homoserine synthesis in the L-threonine biosynthesis pathway. Non-limiting examples of polypeptides with enzyme activities that use and deplete L-homoserine in a bacterium include homoserine kinase, homoserine O-transsuccinylase, and homoserine transacetylase. Non-limiting examples of polypeptides with enzyme activities that use and deplete intermediates that lead to L-homoserine synthesis may include dihydrodipicolinate synthase that depletes aspartyl semialdehyde for L-lysine biosynthesis. In preferred embodiments, a recombinant bacterium may comprise attenuated expression of at least one polypeptide with enzyme activity that uses and depletes L-homoserine. For example, the expression of a nucleic acid encoding a polypeptide with enzyme activity that uses and depletes L-homoserine may be attenuated by at least about 10, about 20, about 30, about 40, about 50, about 60, about 70, about 80, about 90 or about 100% of the wild-type expression. In a preferred embodiment, a recombinant bacterium may comprise attenuated expression of a homoserine kinase. In a particularly preferred embodiment, a recombinant bacterium may comprise attenuated expression of the genomic E. coli thrB nucleic acid encoding homoserine kinase. In an exemplary embodiment, a recombinant bacterium comprising attenuated expression of the thrB nucleic acid encoding a polypeptide with homoserine kinase activity may be the E. coli strain CGSC 8333. In some alternative exemplary embodiments, at least about 10, about 50, about 90 or about 100% of the expression of the thrB nucleic acid may be attenuated. In other alternatives of the exemplary embodiments, at least about 10, about 20, about 30, about 40, about 50, about 60, about 70, about 80, about 90 or about 100% of the expression of the thrB nucleic acid may be attenuated.

A homoserine transport activity may transport the L-homoserine produced in a recombinant bacterium into the medium for easy collection. A homoserine transport activity may also decrease the available concentration of L-homoserine in the cell, therefore decreasing feedback inhibition (as described above). In some embodiments, a recombinant bacterium of the invention may comprise one or more exogenous nucleic acids encoding a polypeptide with homoserine transport activity. Non-limiting examples of exogenous nucleic acids encoding a polypeptide with homoserine transport activity include the E. coli rhtA nucleic acid sequence encoding a threonine and homoserine efflux protein of the DMT family of metabolite export proteins, the rhtB nucleic acid sequence encoding a homoserine/homoserine lactone efflux pump of the RhtB/LysE family of metabolite export proteins, and alleles of rhtA or rhtB that comprise at least one mutation that results in increased expression of the nucleic acid without affecting the structural sequence. In an exemplary embodiment, a recombinant bacterium may comprise the E. coli rhtA exogenous nucleic acid encoding a polypeptide with homoserine transport activity. In another embodiment, a recombinant bacterium may comprise the E. coli rhtA23 exogenous nucleic acid encoding a polypeptide with homoserine transport activity. In another exemplary embodiment, a recombinant bacterium may comprise the E. coli rhtB exogenous nucleic acid encoding a polypeptide with homoserine transport activity.

A recombinant bacterium of the invention may comprise one or more exogenous nucleic acids encoding a polypeptide with activities that increase the availability of precursors used by the L-threonine synthesis pathway. For instance, L-threonine is synthesized from L-aspartate. L-aspartate is synthesized from oxaloacetate which is produced from glucose and the TCA cycle (diagram above). In some embodiments, a recombinant bacterium for producing L-homoserine may comprise one or more exogenous nucleic acids encoding a polypeptide with activities that increase the availability of oxaloacteate. The availability of oxaloacetate may be increased by expressing a nucleic acid sequence encoding phosphoenolpyruvate carboxylase. In an exemplary embodiment, a recombinant bacterium may comprise the E. coli ppc exogenous nucleic acid sequence encoding phosphoenolpyruvate carboxylase.

In an exemplary embodiment, a recombinant bacterium of the invention comprises exogenous nucleic acids encoding polypeptides with aspartokinase activity, homoserine dehydrogenase activity, phosphoenolpyruvate carboxylase activity, and homoserine transport activity, and attenuated expression of the genomic nucleic acid encoding a polypeptide with homoserine kinase activity. In another exemplary embodiment, a recombinant bacterium of the invention comprises exogenous nucleic acids encoding polypeptides with aspartokinase activity, homoserine dehydrogenase activity, and homoserine transport activity, and attenuated expression of the genomic nucleic acid encoding a polypeptide with homoserine kinase activity.

In a particularly exemplary embodiment, a recombinant bacterium is an E. coli bacterium comprising attenuated activity of the genomic thrB nucleic acid sequence encoding homoserine kinase, the mutant E. coli thrA* nucleic acid sequence encoding a polypeptide with dual homoserine dehydrogenase and aspartokinase activities that are free of feedback inhibition, the E. coli rhtA nucleic acid sequence encoding a polypeptide that catalyzes the efflux of L-homoserine into the medium, and the E. coli ppc nucleic acid sequence encoding phosphoenolpyruvate carboxylase.

In another particularly exemplary embodiment, a recombinant bacterium is an E. coli bacterium comprising attenuated activity of the genomic thrB nucleic acid sequence encoding homoserine kinase, the mutant E. coli thrA* nucleic acid sequence encoding a polypeptide with dual homoserine dehydrogenase and aspartokinase activities that are free of feedback inhibition, and the E. coli rhtA nucleic acid sequence encoding a polypeptide that catalyzes the efflux of L-homoserine into the medium.

In yet another particularly exemplary embodiment, a recombinant bacterium is an E. coli bacterium comprising attenuated activity of the genomic thrB nucleic acid sequence encoding homoserine kinase, the mutant E. coli thrA* nucleic acid sequence encoding a polypeptide with dual homoserine dehydrogenase and aspartokinase activities that are free of feedback inhibition, and the E. coli rhtB nucleic acid sequence encoding a polypeptide that catalyzes the efflux of L-homoserine into the medium.

### (b) Promoters

The one or more exogenous nucleic acids of the invention may be operably linked to a promoter. The term “operably-linked”, as used herein, means that expression of a nucleic acid is under the control of a promoter with which it is spatially connected. For instance, in some embodiments, a promoter may be positioned 5′ (upstream) of a nucleic acid under its control. The distance between the promoter and an exogenous nucleic acid it controls may be approximately the same as the native distance between the promoter and the endogenous sequence the promoter regulates. As is known in the art, variation in this distance may be accommodated without loss of promoter function.

The term “promoter”, as used herein, may mean a synthetic or naturally-derived molecule which is capable of conferring, activating or enhancing expression of a nucleic acid in a cell. The promoter may be the native promoter normally associated with a nucleic acid of the invention, or may be a heterologous (e.g. non-native) promoter operably linked to a nucleic acid of the invention. A promoter may comprise one or more specific transcriptional regulatory sequences to further enhance expression and/or to alter the spatial position and/or temporal expression of same.

In some embodiments, the exogenous nucleic acids may be operably linked to a heterologous promoter. Exemplary heterologous promoters include the P_{tac}, P_{trc}, Pₜᵣₚ, P_{lac}, Pₗ and Pᵣ promoters. In some embodiments, the exogenous nucleic acids may be linked to the P_{tac} promoter. In some embodiments, one, two, three, four or more exogenous nucleic acids may be operably linked to the P_{tac} promoter. The sequences of the promoters recited herein are well known in the art.

In preferred embodiments, the exogenous nucleic acids may be operably linked to the native promoter normally associated with a nucleic acid of the invention. In these embodiments, the native promoter is the nucleic acid sequence upstream of the coding region in question which is sufficient for expression of the coding region. In particular embodiments, the native promoter is the nucleic acid sequence upstream of the coding region in question which is both necessary and sufficient for expression of the coding region. The sequences corresponding to native E. coli promoters are well known in the art. In some embodiments, one, two, three, four or more exogenous nucleic acids may be operably linked to the native promoter. In one embodiment, one of the exogenous nucleic acids of the invention may be operably linked to a native promoter. In a preferred embodiment, two of the exogenous nucleic acids of the invention may be linked to a native promoter. In another preferred embodiment, three of the exogenous nucleic acids of the invention may be operably linked to a native promoter.

In a preferred embodiment, when a recombinant bacterium comprises exogenous nucleic acids encoding polypeptides with aspartokinase activity, homoserine dehydrogenase activity, phosphoenolpyruvate activity, and homoserine transport activity, the exogenous nucleic acids encoding polypeptides with phosphoenolpyruvate carboxylase activity and homoserine transport activity are operably linked to a native promoter, and the exogenous nucleic acid encoding aspartokinase activity and homoserine dehydrogenase activity are operably linked to the P_{tac} promoter. In another preferred embodiment, when a recombinant bacterium comprises exogenous nucleic acids encoding polypeptides with aspartokinase activity, homoserine dehydrogenase activity, phosphoenolpyruvate activity, and homoserine transport activity, the exogenous nucleic acids encoding polypeptides with aspartokinase activity, homoserine dehydrogenase activity, and homoserine transport activity are operably linked to a native promoter, and the exogenous nucleic acid encoding phosphoenolpyruvate activity is operably linked to the P_{tac} promoter. In yet another preferred embodiment, when a recombinant bacterium comprises exogenous nucleic acids encoding polypeptides with aspartokinase activity, homoserine dehydrogenase activity, phosphoenolpyruvate activity, and homoserine transport activity, the exogenous nucleic acids encoding polypeptides with aspartokinase activity, homoserine dehydrogenase activity, phosphoenolpyruvate activity, and homoserine transport activity are operably linked to a native promoter.

In an exemplary embodiment, when a recombinant bacterium comprises exogenous nucleic acids encoding polypeptides with aspartokinase activity, homoserine dehydrogenase activity, phosphoenolpyruvate activity, and homoserine transport activity, the exogenous nucleic acid encoding a polypeptide with phosphoenolpyruvate carboxylase activity is the E. coli ppc nucleic acid and is operably linked to the ppc native promoter, the exogenous nucleic acid encoding a homoserine transport activity is the E. coli rhtA nucleic acid and is operably linked to the rhtA native promoter and, the exogenous nucleic acid encoding a polypeptide with aspartokinase activity and homoserine dehydrogenase activity is the E. coli thrA* nucleic acid and is operably linked to the P_{tac} promoter. In another exemplary embodiment, when a recombinant bacterium comprises exogenous nucleic acids encoding polypeptides with aspartokinase activity, homoserine dehydrogenase activity, phosphoenolpyruvate activity, and homoserine transport activity, the exogenous nucleic acid encoding a polypeptide with phosphoenolpyruvate carboxylase activity is the E. coli ppc nucleic acid and is operably linked to the P_{tac} promoter, the exogenous nucleic acid encoding a homoserine transport activity is the E. coli rhtA nucleic acid and is operably linked to the rhtA native promoter and, the exogenous nucleic acid encoding a polypeptide with aspartokinase activity and homoserine dehydrogenase activity is the E. coli thrA* nucleic acid and is operably linked to the native threonine operon promoter. In yet another exemplary embodiment, when a recombinant bacterium comprises exogenous nucleic acids encoding polypeptides with aspartokinase activity, homoserine dehydrogenase activity, phosphoenolpyruvate activity, and homoserine transport activity, the exogenous nucleic acid encoding a polypeptide with phosphoenolpyruvate carboxylase activity is the E. coli ppc nucleic acid and is operably linked to the ppc native promoter, the exogenous nucleic acid encoding a homoserine transport activity is the E. coli rhtA nucleic acid and is operably linked to the rhtA native promoter and, the exogenous nucleic acid encoding a polypeptide with aspartokinase activity and homoserine dehydrogenase activity is the E. coli thrA* nucleic acid and is operably linked to the native threonine operon promoter. In another exemplary embodiment, when a recombinant bacterium comprises exogenous nucleic acids encoding polypeptides with aspartokinase activity, homoserine dehydrogenase activity, phosphoenolpyruvate activity, and homoserine transport activity, the exogenous nucleic acid encoding a polypeptide with phosphoenolpyruvate carboxylase activity is the E. coli ppc nucleic acid and is operably linked to the ppc native promoter, the exogenous nucleic acid encoding a homoserine transport activity is the E. coli rhtB nucleic acid and is operably linked to the rhtB native promoter and, the exogenous nucleic acid encoding a polypeptide with aspartokinase activity and homoserine dehydrogenase activity is the E. coli thrA* nucleic acid and is operably linked to the native threonine operon promoter.

### (c) Nucleic Acid Constructs

The one or more exogenous nucleic acids of the invention may be introduced into a recombinant bacterium of the invention using a vector. As used herein, “vector” refers to an autonomously replicating nucleic acid unit. The present invention may be practiced with any known type of vector, including viral, cosmid, phagemid, phasmid, and plasmid vectors. The most preferred type of vector is a plasmid vector.

As is well known in the art, plasmids and other vectors may be selected so as to control the level of expression of the nucleic acid sequence encoding a polypeptide by controlling the relative copy number of the vector.

In some cases, a high copy number vector might be optimal for producing L-homoserine. A high copy number vector may have at least 31, 35, 40, 45, 50, 55, 60, 65, 70, 75, 80, 85, 90, 95, or 100 copies per bacterial cell. In some embodiments, a high copy number vector may have at least 100, 125, 150, 175, 200, 225, 250, 275, 300, 325, 350, 375, or 400 copies per bacterial cell. Non-limiting examples of high copy number vectors may include a vector comprising the pUC origin of replication (ori) or pFLAG-CTC. In a preferred embodiment, the high copy number vector may be a vector comprising the pUC ori. In another preferred embodiment, the high copy number vector may be pFLAG-CTC.

In other cases, an intermediate copy number vector might be optimal for producing L-homoserine. For instance, an intermediate copy number vector may have at least 10, 11, 12, 13, 14, 15, 16, 17, 18, 19, 20, 21, 22, 23, 24, 25, 26, 27, 28, 29, or 30 copies per bacterial cell. Non-limiting examples of an intermediate copy number vector may include pBR322 and a vector comprising the p15A ori. In an exemplary embodiment, the intermediate copy number vector may be pBR322.

In preferred embodiments, it may be preferable to use a vector with a low copy number such as at least two, three, four, five, six, seven, eight, nine, or ten copies per bacterial cell. Non limiting examples of low copy number vectors include pACYC184 and pSC101. In a preferred embodiment, the low copy number vector may be pACYC184. In another preferred embodiment, the low copy number vector may be pSC101.

As will be appreciated by a skilled artisan, the number of nucleic acids, and their placement within the vector relative to each other, can and will vary. Methods of making a nucleic acid construct of the invention are known in the art. Additional information may be found in Sambrook et al., Molecular Cloning: A Laboratory Manual (New York: Cold Spring Harbor Laboratory Press, 1989).

### (d) Preferred Embodiments

In a preferred embodiment, a recombinant bacterium for producing L-homoserine is an E. coli bacterium comprising attenuated activity of the genomic thrB nucleic acid sequence encoding homoserine kinase and a pBR322 vector comprising the mutant E. coli thrA* nucleic acid sequence encoding a polypeptide with dual homoserine dehydrogenase and aspartokinase activities that are free of feedback inhibition operably linked to the native threonine operon promoter, and the E. coli rhtA nucleic acid sequence encoding a polypeptide that catalyzes the efflux of L-homoserine into the medium operably linked to the rhtA native promoter. In an exemplary embodiment, a recombinant bacterium for producing L-homoserine is the E. coli strain CGSC 8333 comprising the pNI82 plasmid described in the examples. In another exemplary embodiment, a recombinant bacterium for producing L-homoserine is the E. coli strain MG1665 thrB;;Cm comprising the pNI82 plasmid described in the examples.

In another preferred embodiment, a recombinant bacterium for producing L-homoserine is an E. coli bacterium comprising attenuated activity of the genomic thrB nucleic acid sequence encoding homoserine kinase and a pBR322 vector comprising the mutant E. coli thrA* nucleic acid sequence encoding a polypeptide with dual homoserine dehydrogenase and aspartokinase activities that are free of feedback inhibition operably linked to the native threonine operon promoter, and the E. coli rhtB nucleic acid sequence encoding a polypeptide that catalyzes the efflux of L-homoserine into the medium operably linked to the rhtB native promoter. In an exemplary embodiment, a recombinant bacterium for producing L-homoserine is the E. coli strain CGSC 8333 comprising the pNI14 plasmid described in the examples. In another exemplary embodiment, a recombinant bacterium for producing L-homoserine is the E. coli strain MG1665 thrB;;Cm comprising the pNI14 plasmid described in the examples.

In yet another preferred embodiment, a recombinant bacterium for producing L-homoserine is an E. coli bacterium comprising attenuated activity of the genomic thrB nucleic acid sequence encoding homoserine kinase and a pBR322 vector comprising the mutant E. coli thrA* nucleic acid sequence encoding a polypeptide with dual homoserine dehydrogenase and aspartokinase activities that are free of feedback inhibition operably linked to the native threonine operon promoter, the E. coli ppc nucleic acid encoding a polypeptide with phosphoenolpyruvate carboxylase activity linked to the native ppc promoter, and the E. coli rhtA nucleic acid sequence encoding a polypeptide that catalyzes the efflux of L-homoserine into the medium operably linked to the rhtA native promoter. In an exemplary alternative of the embodiment, a recombinant bacterium for producing L-homoserine is the E. coli strain CGSC 8333 comprising the pNI36 plasmid described in the examples. In another exemplary embodiment, a recombinant bacterium for producing L-homoserine is the E. coli strain MG1665 thrB;;Cm comprising the pNI36 plasmid described in the examples.

In an additional preferred embodiment, a recombinant bacterium for producing L-homoserine is an E. coli bacterium comprising attenuated activity of the genomic thrB nucleic acid sequence encoding homoserine kinase and a pBR322 vector comprising the mutant E. coli thrA* nucleic acid sequence encoding a polypeptide with dual homoserine dehydrogenase and aspartokinase activities that are free of feedback inhibition operably linked to the native threonine operon promoter, the E. coli ppc nucleic acid encoding a polypeptide with phosphoenolpyruvate carboxylase activity linked to the native ppc promoter, and the E. coli rhtB nucleic acid sequence encoding a polypeptide that catalyzes the efflux of L-homoserine into the medium operably linked to the rhtB native promoter. In an exemplary alternative of the embodiment, a recombinant bacterium for producing L-homoserine is the E. coli strain CGSC 8333 comprising the pNI18 plasmid described in the examples. In another exemplary embodiment, a recombinant bacterium for producing L-homoserine is the E. coli strain MG1665 thrB;;Cm comprising the pNI18 plasmid described in the examples.

In another preferred embodiment, a recombinant bacterium for producing L-homoserine is an E. coli bacterium comprising attenuated activity of the genomic thrB nucleic acid sequence encoding homoserine kinase and a pBR322 vector comprising the mutant E. coli thrA* nucleic acid sequence encoding a polypeptide with dual homoserine dehydrogenase and aspartokinase activities that are free of feedback inhibition operably linked to the P_{tac} promoter, the E. coli ppc nucleic acid encoding a polypeptide with phosphoenolpyruvate carboxylase activity linked to the native ppc promoter, and the E. coli rhtA nucleic acid sequence encoding a polypeptide that catalyzes the efflux of L-homoserine into the medium operably linked to the native rhtA promoter. In an exemplary alternative of the embodiment, a recombinant bacterium for producing L-homoserine is the E. coli strain CGSC 8333 comprising the pNI65 plasmid described in the examples. In another exemplary embodiment, a recombinant bacterium for producing L-homoserine is the E. coli strain MG1665 thrB;;Cm comprising the pNI65 plasmid described in the examples.

In yet another preferred embodiment, a recombinant bacterium for producing L-homoserine is an E. coli bacterium comprising attenuated activity of the genomic thrB nucleic acid sequence encoding homoserine kinase and a pBR322 vector comprising the mutant E. coli thrA* nucleic acid sequence encoding a polypeptide with dual homoserine dehydrogenase and aspartokinase activities that are free of feedback inhibition operably linked to the P_{tac} promoter, the E. coli ppc nucleic acid encoding a polypeptide with phosphoenolpyruvate carboxylase activity linked to the native ppc promoter, and the E. coli rhtB nucleic acid sequence encoding a polypeptide that catalyzes the efflux of L-homoserine into the medium operably linked to the native rhtB promoter. In an exemplary alternative of the embodiment, a recombinant bacterium for producing L-homoserine is the E. coli strain CGSC 8333 comprising the pNI52 plasmid described in the examples. In another exemplary embodiment, a recombinant bacterium for producing L-homoserine is the E. coli strain MG1665 thrB;;Cm comprising the pNI52 plasmid described in the examples.

In still another preferred embodiment, a recombinant bacterium for producing L-homoserine is an E. coli bacterium comprising attenuated activity of the genomic thrB nucleic acid sequence encoding homoserine kinase and, a pBR322 vector comprising the mutant E. coli thrA* nucleic acid sequence encoding a polypeptide with dual homoserine dehydrogenase and aspartokinase activities that are free of feedback inhibition operably linked to the native threonine operon promoter, the E. coli ppc nucleic acid encoding a polypeptide with phosphoenolpyruvate carboxylase activity operably linked to the P_{tac}, and the E. coli rhtA nucleic acid sequence encoding a polypeptide that catalyzes the efflux of L-homoserine into the medium operably linked to the native rhtA promoter. In an exemplary alternative of the embodiment, a recombinant bacterium for producing L-homoserine is the E. coli strain CGSC 8333 comprising the pNI66 plasmid described in the examples. In another exemplary embodiment, a recombinant bacterium for producing L-homoserine is the E. coli strain MG1665 thrB;;Cm comprising the pNI66 plasmid described in the examples.

In another preferred embodiment, a recombinant bacterium for producing L-homoserine is an E. coli bacterium comprising attenuated activity of the genomic thrB nucleic acid sequence encoding homoserine kinase and, a pBR322 vector comprising the mutant E. coli thrA* nucleic acid sequence encoding a polypeptide with dual homoserine dehydrogenase and aspartokinase activities that are free of feedback inhibition operably linked to the native threonine operon promoter, the E. coli ppc nucleic acid encoding a polypeptide with phosphoenolpyruvate carboxylase activity operably linked to the P_{tac}, and the E. coli rhtB nucleic acid sequence encoding a polypeptide that catalyzes the efflux of L-homoserine into the medium operably linked to the native rhtB promoter. In an exemplary alternative of the embodiment, a recombinant bacterium for producing L-homoserine is the E. coli strain CGSC 8333 comprising the pNI53 plasmid described in the examples. In another exemplary embodiment, a recombinant bacterium for producing L-homoserine is the E. coli strain MG1665 thrB;;Cm comprising the pNI53 plasmid described in the examples.

### II. Methods of Use

Another aspect of the invention provides a method of producing L-homoserine by cultivating a recombinant bacterium described in section (I) above in a culture medium to produce and secrete L-homoserine into the medium, and collecting the L-homoserine from the medium.

Methods of cultivating a bacterium, and collecting and purifying L-homoserine from the medium are well known in the art and may be similar to conventional fermentation methods for production of an amino acid. The methods are described below.

### (a) Culture Conditions

As will be appreciated by a skilled artisan, the culture conditions for producing L-homoserine can and will vary. A recombinant bacterium may be cultured in a medium comprising a carbon source, a nitrogen source, and minerals, and if necessary, appropriate amounts of nutrients which the bacterium requires for growth. As the carbon source, saccharides such as glucose, fructose, sucrose, molasses and starch hydrolysate, organic acids such as fumaric acid, citric acid and succinic acid, or alcohol such as ethanol and glycerol may be used. As the nitrogen source, various ammonium salts such as ammonia and ammonium sulfate, other nitrogen compounds such as amines, a natural nitrogen source such as peptone, soybean-hydrolysate, or digested fermentative microorganism may be used. As minerals, potassium monophosphate, magnesium sulfate, sodium chloride, ferrous sulfate, manganese sulfate, calcium chloride, and the like may be used. As vitamins, thiamine, yeast extract, and the like, may be used. The pH of the medium may be between about 5 and about 9. In some embodiments where the bacterium comprises a mutation that limits the production of L-threonine such as the thrB deletion, the medium may be supplemented with L-threonine to maintain growth of the bacterium. In an exemplary embodiment, a recombinant bacterium of the invention is cultivated in a medium comprising the MMI medium comprising 30 g/L glucose, 40 g/L CaCO₃, 10 g/L (NH₄)₂SO₄, 1 g/L KH₂PO₄, 1 g/L MgSO₄.7H₂O, 10 mg/L FeSO₄.7H₂O, 10 mg/L MnSO₄.H₂O, 1 mg/L thiamine, 200 mg/L threonine at a pH of about 7.4 as described in Example 1 to produce L-homoserine. In another exemplary embodiment, a recombinant bacterium of the invention is cultivated in a medium comprising the MMII medium comprising 60 g/L glucose, 40 g/L CaCO₃, 20 g/L (NH₄)₂SO₄, 1 g/L KH₂PO₄, 1 g/L MgSO₄.7H₂O, 10 mg/L FeSO₄.7H₂O, 10 mg/L MnSO₄.H₂O, 2 mg/L thiamine, 400 mg/L threonine at a pH of about 7.4 as described in Example 1 to produce L-homoserine.

In essence, various methods of cultivating, including temperature of cultivation and duration of cultivation may be used. The cultivation may be performed under aerobic conditions, such as by shaking and/or stirring with aeration. In some embodiments, a recombinant bacterium of the invention may be cultivated at a temperature of about 25 to about 40° C. In other embodiments, a recombinant bacterium of the invention may be cultivated at a temperature of about 25, 26, 27, 28, 29, 30, 31, 32, 33, 34, 35, 36, 37, 38, 39, and about 40° C. In preferred embodiments, a recombinant bacterium of the invention may be cultivated at a temperature of about 32° C.

A recombinant bacterium of the invention may be cultivated for about 1, 2, 3, 4, 5, 6, 7, 8, 9, 10 or more days before collecting L-homoserine from the medium. In some embodiments, a recombinant bacterium of the invention may be cultivated for about 1 day before collecting L-homoserine from the medium. In other embodiments, a recombinant bacterium of the invention may be cultivated for about 2 days before collecting L-homoserine from the medium. In preferred embodiments, a recombinant bacterium of the invention may be cultivated for about 3 days before collecting L-homoserine from the medium.

### (b) Collection of L-Homoserine

Methods of collecting amino acids such as L-homoserine from culture media are known in the art. After cultivation, solids such as cells may be removed from the liquid medium using separation methods known in the art, such as centrifugation, membrane filtration, decantation, or a combination thereof. The liquid medium may then be concentrated by methods known in the art such as, with the aid of a rotary evaporator, thin-film evaporator, falling-film evaporator, by reverse osmosis or by nanofiltration. The L-amino acid may then be collected and purified by alcohol precipitation or ion-exchange chromatography using a suitable resin as described by Nagai, H. et al., Separation Science and Technology, 39(16), 3691-3710. The purified amino acid may be further concentrated and purified until the desired level of purity and concentration are reached. Concentration separation and purification methods may be as described in the Japanese Patent Laid-open Nos. 9-164323 and 9-173792 and in WO 2008/078448 and WO 2008/078646, all of which are incorporated herein by reference in their entirety.

The yield and purity of L-homoserine produced using a recombinant bacteria of the invention can and will vary depending on the exogenous nucleic acid, the polypeptides encoded by the exogenous nucleic acids and the culture conditions. In some embodiments, a recombinant bacterium of the invention may produce about 2, 5, 15, 20, 25, 30, 35, 40, 45, 50, 55, 60, 65, 70, 75, 80, 85 or more grams of L-homoserine in a liter of culture medium. In other embodiments, a recombinant bacterium of the invention may produce about 2, 5, 30, 35, 40 or more grams of L-homoserine in a liter of culture medium. In still other embodiments, a recombinant bacterium of the invention may produce about 45, 50, 55, 60, 65, 70, 75 or more grams of L-homoserine in a liter of culture medium. Purity of the collected L-amino acid may be, for example, 50% or higher, 85% or higher, or even 95% or higher.

### EXAMPLES

The following examples illustrate various iterations of the invention.

### Example 1

### L-Homoserine Production Using thrA* Expression from an Intermediate Copy Plasmid

The thrA gene encodes a bifunctional enzyme with aspartokinase and homoserine dehydrogenase (AKI-HdHI) activities. Both activities function in L-homoserine synthesis from aspartate in the threonine biosynthesis pathway; aspartokinase converts aspartate to aspartyl phosphate and homoserine dehydrogenase converts aspartic semialdehyde to L-homoserine. In an attempt to increase production of L-homoserine, the thrA gene was expressed from a plasmid in bacteria.

The thrA coding region (SEQ ID NO 2) and its upstream regulatory region (SEQ ID NO 1), as well as the transcriptional terminator region (SEQ ID NO 3) of the threonine operon were amplified by PCR, and inserted into the EcoRI and SphI sites of the pBR322 intermediate copy number plasmid to generate recombinant plasmid pNI1.7. The thrA sequences were amplified from E. coli strain ATCC21277, where the thrA gene encodes a mutated aspartokinase-homoserine dehydrogenase gene, thrA*. The thrA* mutation encodes an aspartokinase-homoserine dehydrogenase that is resistant to feedback inhibition by threonine and isoleucine to maximize L-homoserine production. All sequences used in these examples are listed in Table 6 below.

The pNI1.7 plasmid was transformed into E. coli CGSC 8333 for L-homoserine production. E. coli CGSC 8333 comprises a deletion of the thrB gene which converts L-homoserine to L-threonine. Such a mutation increases the accumulation and production of L-homoserine. The transformed bacterium and a control bacterium devoid of the pNI1.7 plasmid were cultured in 100 ml minimal media as described in Table 1. Minimal medium I was supplemented with 30 g/L glucose, 10 g/L (NH₄)SO₄, 1 mg/L thiamine and 200 mg/L threonine, whereas minimal medium II was supplemented with 60 g/L glucose, 20 g/L (NH₄)SO₄, 2 mg/L thiamine and 400 mg/L threonine. The pH of both media was 7.4. The cultures were grown in 500 ml baffled flasks and shaken at 225 rpm at 30° C., 32° C., 35° C., or 37° C. Samples were taken at 24, 48, and 72 h and L-homoserine concentration determined in the supernatants using HPLC.

TABLE 1

| | Amount/L | | | |
|---|---|---|---|---|
| Component | MMI | | MMII | |
| Glucose | 30 | g | 60 | g |
| CaCO₃ | 40 | g | 40 | g |
| (NH₄)2SO₄ | 10 | g | 20 | g |
| KH2PO₄ | 1 | g | 1 | g |
| MgSO₄•7H₂O | 1 | g | 1 | g |
| FeSO₄•7H₂O | 10 | mg | 10 | mg |
| MnSO₄•H₂O | 10 | mg | 10 | mg |
| Thiamine | 1 | mg | 2 | mg |

| | | | |
|---|---|---|---|
| Threonine | 200 | mg | 400 |

| | |
|---|---|
| pH | 7.4 |

MMI: Minimal Medium I; MMII: Minimal Medium II

Other than pBR322 (GenBank Accession #J01749), low copy number plasmids, such as, pACYC184 (GenBank Accession #X06403), and pSC101 (GenBank Accession # X01654), and high copy number plasmids, such as pUC19 (GenBank Accession #L09137) and pFLAG-CTC (Sigma-Aldrich Product No. E8408, sequence No. E5394) were also used. Optimal results were obtained with the pBR322 intermediate copy plasmid, and that plasmid was therefore used for further experiments. Culturing bacteria at 32° C. produced the best result; the CGSC 8333 bacterium comprising the pNI1.7 plasmid produced 3.5, 3.6, 2.5, and 2.3 g/L homoserine in 100 mL minimal medium I in 500 mL baffled shake flasks in 72 h at 30, 32, 35, and 37° C. respectively. The 32° C. culture temperature was used for further experiments. In another experiment where the bacteria were cultured at 32° C., the control CGSC 8333 E. coli only produced 1.5 g/L of L-homoserine in the supernatant, whereas the CGSC 8333 bacterium comprising the pNI1.7 plasmid produced 4.9 g/L of L-homoserine (See Example 2 below). In a 1L fermentor, the CGSC 8333 bacterium comprising the pNI1.7 plasmid produced 19.7 g/L homoserine in the supernatant in 72 h at 32° C.

### Example 2

### Expression of thrA*, ppc, rhtA and rhtB for L-Homoserine Production

L-threonine, which belongs to the aspartic acid family of amino acids, is synthesized from L-aspartate. The ppc gene encodes phosphoenolpyruvate carboxylase which catalyzes the conversion of phosphoenolpyruvate to oxaloacetate, which is then converted to aspartate. The rhtA and rhtB genes encode membrane proteins belonging to the drug metabolite transporter superfamily and export L-homoserine and threonine out of the cell.

To further enhance L-homoserine production, the ppc and rhtA or rhtB coding regions were also cloned and expressed in E. coli with the thrA* gene. The ppc coding region and its upstrean and downstream regulatory regions (SEQ ID NO 4) were amplified from the CGSC 8333 genome and cloned in the SalI and EagI sites of pNI1.7 described in Example 1 above, to obtain the pNI10 plasmid. The rhtA coding region and 5′ and 3′ regulatory regions (SEQ ID NO 5) were amplified and cloned into the EagI and NruI sites of pNI1.7 to generate the pNI82 plasmid. The rhtB coding region and its upstream regulatory region and downstream transcriptional terminator region were amplified and cloned into the EagI and NruI sites of pNI1.7 to generate the pNI14 plasmid. In addition, plasmid pNI18 was generated to express thrA*, ppc and rhtB from their native promoters. The ppc coding region and its upstrean and downstream sequences and the rhtA coding region and 5′ and 3′ regulatory regions were also both cloned into pNI1.7 to produce the pNI36 plasmid to express thrA*, ppc and rhtA. pNI1.7, pNI10, pNI82 and pNI36 were transformed into E. coli strain CCGSC 8333 and cultured in 100 ml minimal medium I or minimal medium II (Table 1), in 500 ml baffled flasks shaken at 200 rpm, at 32° C. Samples were taken at 24, 48, and 72 h and L-homoserine concentration was determined in the supernatants using HPLC.

Under the above conditions, L-homoserine production in the various bacteria comprising the constructed plasmids is summarized in Table 2. The CGSC 8333 host alone produced 1.5 g/L and 1.2 g/L L-homoserine. CGSC 8333 transformed with pNI1.7 plasmid expressing thrA* alone produced 4.9 g/L and 6.2 g/L of L-homoserine. CGSC 8333 transformed with the pNI10 plasmid expressing thrA* and ppc produced 4.0 and 3.2 g/L L-homoserine. CGSC 8333 transformed with the pNI82 plasmid expressing thrA* and rhtA produced 15.3 and 29.2 g/L L-homoserine. CGSC 8333 transformed with the pNI36 plasmid expressing thrA*, ppc and rhtA produced 18.7 and 33.9 g/L L-homoserine.

The highest producing plasmid/strain (CGSC 8333 transformed with the pNI36 plasmid) was tested further in a 1 L fermentor, where the CGSC 8333 bacterium comprising the pNI36 plasmid produced 73 g/L in the supernatant after 72 h fermentation at 32° C.

TABLE 2A
Plasmids

| Plasmid | Genes cloned |
|---|---|
| pNI1.7 | thrA* |
| pNI10 | thrA*, ppc |
| pNI14 | thrA*, rhtB |
| pNI18 | thrA*, ppc, rhtB |
| pNI82 | thrA*, rhtA |
| pNI36 | thrA*, ppc, rhtA |

TABLE 2B
Homoserine Production

| | | Homoserine (g/L) | |
|---|---|---|---|
| Plasmid | Strain | MMI | MMII |
| | 8333 | 1.5 | 1.2 |
| pNI1.7 | 8333 | 4.9 | 6.2 |
| pNI10 | 8333 | 4.0 | 3.2 |
| pNI14 | 8333 | 4.7 | — |
| pNI82 | 8333 | 15.3 | 29.2 |
| pNI36 | 8333 | 18.7 | 33.9 |

### Example 3

### Native Promoters Induce Higher Levels of L-Homoserine Production than Strong Promoters

To determine if overexpression of the thrA, ppc and rhtA genes using non-native, strong promoters would increase L-homoserine production in E. coli, the native promoters of thrA, ppc, and rhtA genes in pNI36 were replaced individually or in combination with the tac promoter (P_{tac}) derived from the lac UV5 promoter.

First, thrA*, ppc and rhtA expression cassettes controlled by the P_{tac} promoter were generated. The thrA*, ppc, and rhtA coding regions were each cloned into the multiple cloning site of the pFLAG-CTC plasmid downstream of the P_{tac} promoter sequence. The resulting P_{tac}-controlled expression cassettes comprising the thrA*, ppc, or rhtA genes, as well as the thrA*, ppc, or rhtA expression cassettes described in the previous examples controlled by the native promoters of each gene, were amplified by PCR and sub-cloned into pBR322. The resulting plasmids comprising the various combinations of thrA*, ppc, or rhtA controlled by the respective native promoter or the P_{tac} promoter are listed in Table 3 below.

All plasmids listed in Table 3 were transformed into CGSC 8333 and cultured in 500 ml baffled flasks as described above. Cultures comprising plasmids with the P_{tac} promoter were supplemented with 1 mM IPTG after 24 h culture to induce P_{tac} controlled expression, and then cultured for 72 h to produce L-homoserine. Cultures comprising the pNI36 plasmid were directly cultured for 72 h without IPTG induction. The culture temperature for all of the strains was 32° C. The results are listed in Table 3.

TABLE 3A
Comparison of native and non-native promoters for
L-homoserine production (rhtA).

| | Genes controlled by Ptac | | | Homoserine (g/L) | |
|---|---|---|---|---|---|
| Plasmid | thrA* | ppc | rhtA | MMI | MMII |
| pNI18 | | | | 18.7 | |
| pNI64 | X | X | | 5.4 | |
| pNI65 | X | | | 14.5 | |
| pNI66 | | X | | 10.3 | |
| pNI68 | X | X | X | 1.7 | |
| pNI69 | X | | X | 1.5 | |
| pNI70 | | X | X | 1.8 | |
| pNI71 | | | X | 2.7 | |

X denotes a gene controlled by the P_{tac} promoter.
Blank cells denote a gene controlled by its native promoter.

TABLE 3B
Comparison of native and non-native
promoters for L-homoserine production (rhtB).

| | Genes controlled by Ptac | | |
|---|---|---|---|
| Plasmid | thrA* | ppc | rhtB |
| pNI36 | | | |
| pNI52 | X | | |
| pNI53 | | X | |

The results indicate that when P_{tac} replaced the native promoter of a gene, L-homoserine production was decreased. This is especially true when the native promoter of rhtA is replaced with the P_{tac} promoter.

### Example 4

### Expression of AKI-HdHI and Stability of P_{tac} Plasmid

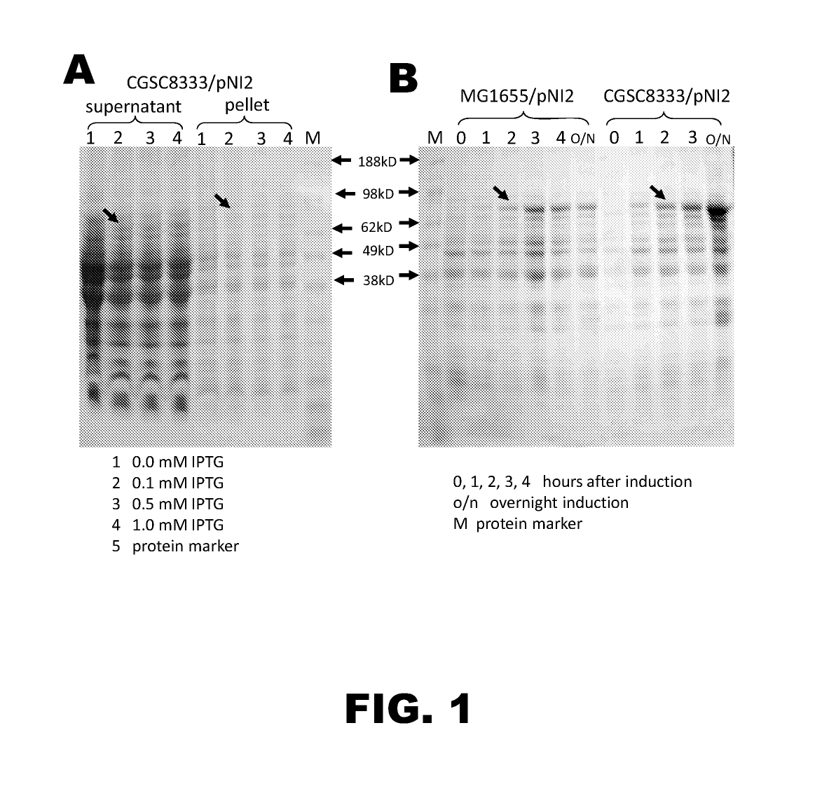

When induced with 0.1-1.0 mM/ml IPTG, P_{tac}-controlled thrA* over-expressed considerable amounts of protein in the cell (FIG. 1). CGSC 8333 bacteria were transformed with pNI2, a plasmid comprising the pFLAG-CTC expression vector carrying the thrA* coding region downstream of the Ptac promoter cloned at the NdeI and SalI sites. AKI-HdHI expression was then induced with 0.1, 0.5, and 1 mM IPTG for 2 h. Cells were lysed, and fractionated into a soluble supernatant fraction, and an insoluble pellet fraction. Proteins in the soluble and insoluble fractions were separated on an SDS-PAGE gel. A 90 kD AKI-HdHI protein band was present in both soluble and insoluble samples (FIG. 1A).

Expression of AKI-HdHI was also measured in MG1655 and CGSC8333 bacterial strains transformed with the pNI2 plasmid and induced with IPTG for various durations. Only the soluble fraction of the extracts was examined (FIG. 1B). The expression level of 90 kD protein varied with the duration of induction. The highest expression occurred at 3 h in the MG1655 strain, but overnight incubation produced the highest expression in the CGSC 8333 strain.

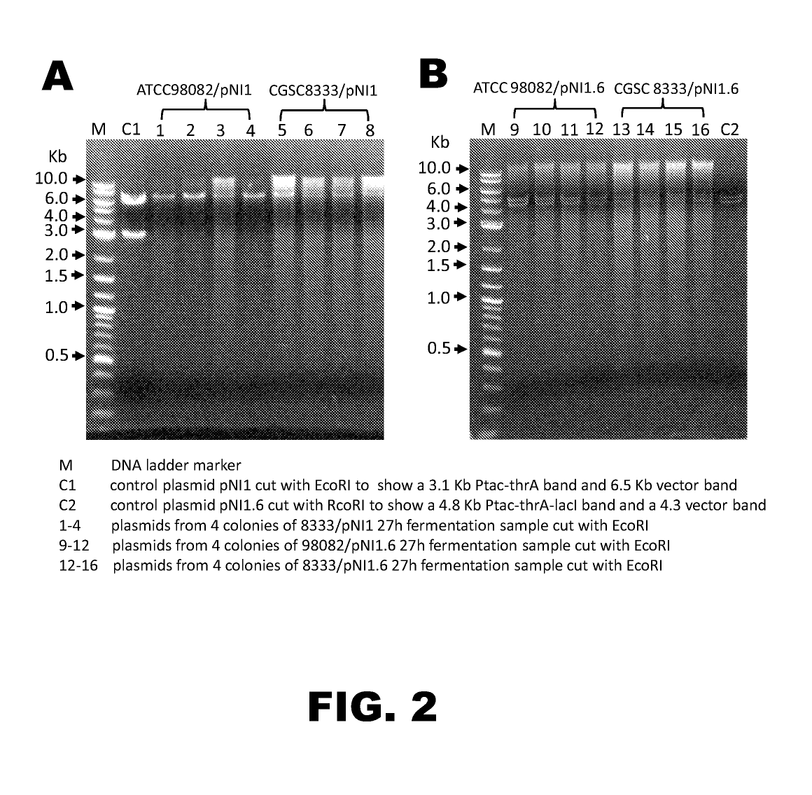

The constructs harboring the P_{tac}-thrA* cassette were not stable in E. coli unless the lacI repressor gene (SEQ ID NO 6) was also present in the same construct (FIG. 2). ATCC98082 and CGSC8333 E. coli K-12 strains were transformed with pNI1, a plasmid comprising the pBR322 plasmid carrying the thrA* coding region downstream of the P_{tac} promoter. The cultures were grown in 3 L fermentors in minimal media as described above, at 32° C. Fermation samples were collected at 27 h, and plated on LB agar plates supplemented with 100 μg/mL Ampicillin. After incubation of the plates, 4 individual colonies were picked and further cultured in LB liquid medium with 100 μg/mL Ampicillin for plasmid isolation. Plasmid DNA was then digested with EcoRI, and the resulting digested DNA was separated on an agarose gel. The results show the absence of the P_{tac}-thrA*band in every fermentation sample compared to the intact control plasmid in Lane 2. Conversely, when a similar experiment was performed with the pNI1.6 plasmid comprising pBR322 carrying the P_{tac}-thrA*expression construct and the lacI gene, the specific P_{tac}-thrA*band was not lost after culture. Therefore, lacI might prevent thrA* from being deleted from the vector during fermentation.

### Example 5

### Testing Additional Strains of E. coli with pNI 36 to Produce Homoserine

To determine the effect of pNI36 on L-homoserine production in E. coli strains other than CGSC 8333, three additional E. coli strains were constructed: MG1655 thrB::Cm, E. coli B WT thrB::Cm, and E. coli B REL606 thrB::Cm. In the 3 new strains, the thrB gene was replaced with chlorampenicol acetyltransferase gene (cat). Cells were then transformed with the pNI 36 construct (Table 4), and grown in shake flasks as described above. In short, the bacteria were grown in 100 ml of minimal media II (Table 1). The cells were grown at 225 RPM and 32° C. for 72 hours. One ml samples were collected, filtered, and L-homoserine production was measured by HPLC (Table 4).

TABLE 4
Comparison of different E. coli strains transformed with pNI36.

| Strain | Homoserine g/L (72 h) |
|---|---|
| MG1655 thrB::Cm | 0.807 |
| MG1655 thrB::Cm/pNI36 | 26.9 |
| E. coli B thrB::Cm | 0.844 |
| E. coli B thrB::Cm/pNI36 | 12.6 |
| E. coli B REL606 thrB::Cm | 1.094 |
| E. coli B REL606 thrB::Cm/pNI36 | 31.9 |

The results clearly indicate that pNI 36 can increase homoserine production in two E. coli B strains and the wildtype K-12 strain MG1655. Further cultures grown in 3 L fermentors in minimal media as described above, at 32° C., also showed good overall productivity (Table 5).

TABLE 5
Comparison of different E. coli strains grown at the 3L scale.

| Strain | Homoserine g/L (72 h) |
|---|---|
| GGSC 8333/pNI18 | 51.066 |
| MG1655/pNI36 | 67.754 |
| MG1655 thrB::Cm/pNI36 | 71.07 |
| E. coli B REL606 thrB::Cm | 56.969 |
| GGSC 8333/pNI36 | 73.069 |

TABLE 6
Sequences used in the studies described above.

SEQ ID NO 1. upstream region of thrA

| | |
|---|---|
| 1 | AGCTTTTCAT TCTGACTGCA ACGGGCAATA TGTCTCTGTG TGGATTAAAA AAAGAGTGTC |
| 61 | TGATAGCAGC TTCTGAACTG GTTACCTGCC GTGAGTAAAT TAAAATTTTA TTGACTTAGG |
| 121 | TCACTAAATA CTTTAACCAA TATAGGCATA GCGCACAGAC AGATAAAAAT TACAGAGTAC |
| 181 | ACAACATCCA TGAAACGCAT TAGCACCACC ATTACCACCA CCATCACCAT TACCACAGGT |
| 241 | AACGGTGCGG GCTGACGCGT ACAGGAAACA CAGAAAAAAG CCCGCACCTG ACAGTGCGGG |
| 301 | CTTTTTTTTT CGACCAAAGG TAACGAGGTA ACAACC |

SEQ ID NO 2. thrA coding region

| | |
|---|---|
| 1 | ATGCGAGTGT TGAAGTTCGG CGGTACATCA GTGGCAAATG CAGAACGTTT TCTGCGTGTT |
| 61 | GCCGATATTC TGGAAAGCAA TGCCAGGCAG GGGCAGGTGG CCACCGTCCT CTCTGCCCCC |
| 121 | GCCAAAATCA CCAACCACCT GGTGGCGATG ATTGAAAAAA CCATTAGCGG CCAGGATGCT |
| 181 | TTACCCAATA TCAGCGATGC CGAACGTATT TTTGCCGAAC TTTTGACGGG ACTCGCCGCC |
| 241 | GCCCAGCCGG GGTTCCCGCT GGCGCAATTG AAAACTTTCG TCGATCAGGA ATTTGCCCAA |
| 301 | ATAAAACATG TCCTGCATGG CATTAGTTTG TTGGGGCAGT GCCCGGATAG CATCAACGCT |
| 361 | GCGCTGATTT GCCGTGGCGA GAAAATGTCG ATCGCCATTA TGGCCGGCGT ATTAGAAGCG |
| 421 | CGCGGTCACA ACGTTACTGT TATCGATCCG GTCGAAAAAC TGCTGGCAGT GGGGCATTAC |
| 481 | CTCGAATCTA CCGTCGATAT TGCTGAGTCC ACCCGCCGTA TTGCGGCAAG CCGCATTCCG |
| 541 | GCTGATCACA TGGTGCTGAT GGCAGGTTTC ACCGCCGGTA ATGAAAAAGG CGAACTGGTG |
| 601 | GTGCTTGGAC GCAACGGTTC CGACTACTCT GCTGCGGTGC TGGCTGCCTG TTTACGCGCC |
| 661 | GATTGTTGCG AGATTTGGAC GGACGTTGAC GGGGTCTATA CCTGCGACCC GCGTCAGGTG |
| 721 | CCCGATGCGA GGTTGTTGAA GTCGATGTCC TACCAGGAAG CGATGGAGCT TTCCTACTTC |
| 781 | GGCGCTAAAG TTCTTCACCC CCGCACCATT ACCCCCATCG CCCAGTTCCA GATCCCTTGC |
| 841 | CTGATTAAAA ATACCGGAAA TCCTCAAGCA CCAGGTACGC TCATTGGTGC CAGCCGTGAT |
| 901 | GAAGACGAAT TACCGGTCAA GGGCATTTCC AATCTGAATA ACATGGCAAT GTTCAGCGTT |
| 961 | TCTGGTCCGG GGATGAAAGG GATGGTCGGC ATGGCGGCGC GCGTCTTTGC AGCGATGTCA |
| 1021 | CGCGCCCGTA TTTCCGTGGT GCTGATTACG CAATCATCTT CCGAATACAG CATCAGTTTC |
| 1081 | TGCGTTCCAC AAAGCGACTG TGTGCGAGCT GAACGGGCAA TGCAGGAAGA GTTCTACCTG |
| 1141 | GAACTGAAAG AAGGCTTACT GGAGCCGCTG GCAGTGACGG AACGGCTGGC CATTATCTCG |
| 1201 | GTGGTAGGTG ATGGTATGCG CACCTTGCGT GGGATCTCGG CGAAATTCTT TGCCGCACTG |
| 1261 | GCCCGCGCCA ATATCAACAT TGTCGCCATT GCTCAGGGAT CTTCTGAACG CTCAATCTCT |
| 1321 | GTCGTGGTAA ATAACGATGA TGCGACCACT GGCGTGCGCG TTACTCATCA GATGCTGTTC |
| 1381 | AATACCGATC AGGTTATCGA AGTGTTTGTG ATTGGCGTCG GTGGCGTTGG CGGTGCGCTG |
| 1441 | CTGGAGCAAC TGAAGCGTCA GCAAAGCTGG CTGAAGAATA AACATATCGA CTTACGTGTC |
| 1501 | TGCGGTGTTG CCAACTCGAA GGCTCTGCTC ACCAATGTAC ATGGCCTTAA TCTGGAAAAC |
| 1561 | TGGCAGGAAG AACTGGCGCA AGCCAAAGAG CCGTTTAATC TCGGGCGCTT AATTCGCCTC |
| 1621 | GTGAAAGAAT ATCATCTGCT GAACCCGGTC ATTGTTGACT GCACTTCCAG CCAGGCAGTG |
| 1681 | GCGGATCAAT ATGCCGACTT CCTGCGCGAA GGTTTCCACG TTGTCACGCC GAACAAAAAG |
| 1741 | GCCAACACCT CGTCGATGGA TTACTACCAT CAGTTGCGTT ATGCGGCGGA AAAATCGCGG |
| 1801 | CGTAAATTCC TCTATGACAC CAACGTTGGG GCTGGATTAC CGGTTATTGA GAACCTGCAA |
| 1861 | AATCTGCTCA ATGCAGGTGA TGAATTGATG AAGTTCTCCG GCATTCTTTC TGGTTCGCTT |
| 1921 | TCTTATATCT TCGGCAAGTT AGACGAAGGC ATGAGTTTCT CCGAGGCGAC CACGCTGGCG |
| 1981 | CGGGAAATGG GTTATACCGA ACCGGACCCG CGAGATGATC TTTCTGGTAT GGATGTGGCG |
| 2041 | CGTAAACTAT TGATTCTCGC TCGTGAAACG GGACGTGAAC TGGAGCTGGC GGATATTGAA |
| 2101 | ATTGAACCTG TGCTGCCCGC AGAGTTTAAC GCCGAGGGTG ATGTTGCCGC TTTTATGGCG |
| 2161 | |

SEQ ID NO 3. Transcriptional terminator of threonine operon

| | |
|---|---|
| 1 | AATCTATTCA TTATCTCAAT CAGGCCGGGT TTGCTTTTAT GCAGCCCGGC TTTTTTATGA |
| 61 | AGAAATTATG GAGAAAAATG ACAGGGAAAA AGGAGAAATT CTCAATAAAT GCGGTAACTT |
| 121 | AGAGATTAGG ATTGCGGAGA ATAACAACCG CCGTTCTCAT CGAGTAATCT CCGGATATCG |
| 181 | ACCCATAACG GGCAATGATA AAAGGAGTAA CCTGTGAAAA AGATGCAATC TATCGTACTC |
| 241 | GCACTTTCCC TGGTTCTGGT CGCTCCCATG GCAGCACAGG CTGCGGAAAT TACGTTAGTC |
| 301 | CCGTCAGTAA AATTACAGAT AGGCGATCGT GATAATCGTG GCTATTACTG GGATGGAGGT |
| 361 | CACTGGCGCG ACCACGGCTG GTGGAAACAA CATTATGAAT GGCGAGGCAA TCGCTGGCAC |
| 421 | CTACACGGAC CGCCGCCACC GCCGCGCCAC CATAAGAAAG CTCCTCATGA TCATCACGGC |
| 481 | GGTCATGGTC CAGGCAAACA TCACCGCTAA ATGACAAATG CCGGGTAACA ATCCGGCATT |
| 541 | CAGCGCCTGA TGCGACGCTG GCGCGTCTTA TCAGGCCTAC GTTAATTCTG CAATATATTG |
| 601 | AATCTGCATG CTTTTGTAGG CAGGATAAGG CGTTCACGCC GCATCCGGCA TTGACTGCAA |
| 661 | ACTTA |

SEQ ID NO 4. ppc coding region and upstream and downstream
regulatory regions (ppc translational start and stop
codons are in bold)

| | |
|---|---|
| 1 | TGCTGAAGCG ATTTCGCAGC ATTTGACGTC ACCGCTTTTA CGTGGCTTTA TAAAAGACGA |
| 61 | CGAAAAGCAA AGCCCGAGCA TATTCGCGCC AATGCGACGT GAAGGATACA GGGCTATCAA |
| 121 | ACGATAAGAT GGGGTGTCTG GGGTAATATG AACGAACAAT ATTCCGCATT GCGTAGTAAT |
| 181 | GTCAGTATGC TCGGCAAAGT GCTGGGAGAA ACCATCAAGG ATGCGTTGGG AGAACACATT |
| 241 | CTTGAACGCG TAGAAACTAT CCGTAAGTTG TCGAAATCTT CACGCGCTGG CAATGATGCT |
| 301 | AACCGCCAGG AGTTGCTCAC CACCTTACAA AATTTGTCGA ACGACGAGCT GCTGCCCGTT |
| 361 | GCGCGTGCGT TTAGTCAGTT CCTGAACCTG GCCAACACCG CCGAGCAATA CCACAGCATT |
| 421 | TCGCCGAAAG GCGAAGCTGC CAGCAACCCG GAAGTGATCG CCCGCACCCT GCGTAAACTG |
| 481 | AAAAACCAGC CGGAACTGAG CGAAGACACC ATCAAAAAAG CAGTGGAATC GCTGTCGCTG |
| 541 | GAACTGGTCC TCACGGCTCA CCCAACCGAA ATTACCCGTC GTACACTGAT CCACAAAATG |
| 601 | GTGGAAGTGA ACGCCTGTTT AAAACAGCTC GATAACAAAG ATATCGCTGA CTACGAACAC |
| 661 | AACCAGCTGA TGCGTCGCCT GCGCCAGTTG ATCGCCCAGT CATGGCATAC CGATGAAATC |
| 721 | CGTAAGCTGC GTCCAAGCCC GGTAGATGAA GCCAAATGGG GCTTTGCCGT AGTGGAAAAC |
| 781 | AGCCTGTGGC AAGGCGTACC AAATTACCTG CGCGAACTGA ACGAACAACT GGAAGAGAAC |
| 841 | CTCGGCTACA AACTGCCCGT CGAATTTGTT CCGGTCCGTT TTACTTCGTG GATGGGCGGC |
| 901 | GACCGCGACG GCAACCCGAA CGTCACTGCC GATATCACCC GCCACGTCCT GCTACTCAGC |
| 961 | CGCTGGAAAG CCACCGATTT GTTCCTGAAA GATATTCAGG TGCTGGTTTC TGAACTGTCG |
| 1021 | ATGGTTGAAG CGACCCCTGA ACTGCTGGCG CTGGTTGGCG AAGAAGGTGC CGCAGAACCG |
| 1081 | TATCGCTATC TGATGAAAAA CCTGCGTTCT CGCCTGATGG CGACACAGGC ATGGCTGGAA |
| 1141 | GCGCGCCTGA AAGGCGAAGA ACTGCCAAAA CCAGAAGGCC TGCTGACACA AAACGAAGAA |
| 1201 | CTGTGGGAAC CGCTCTACGC TTGCTACCAG TCACTTCAGG CGTGTGGCAT GGGTATTATC |
| 1261 | GCCAACGGCG ATCTGCTCGA CACCCTGCGC CGCGTGAAAT GTTTCGGCGT ACCGCTGGTC |
| 1321 | CGTATTGATA TCCGTCAGGA GAGCACGCGT CATACCGAAG CGCTGGGCGA GCTGACCCGC |
| 1381 | TACCTCGGTA TCGGCGACTA CGAAAGCTGG TCAGAGGCCG ACAAACAGGC GTTCCTGATC |
| 1441 | CGCGAACTGA ACTCCAAACG TCCGCTTCTG CCGCGCAACT GGCAACCAAG CGCCGAAACG |
| 1501 | CGCGAAGTGC TCGATACCTG CCAGGTGATT GCCGAAGCAC CGCAAGGCTC CATTGCCGCC |
| 1561 | TACGTGATCT CGATGGCGAA AACGCCGTCC GACGTACTGG CTGTCCACCT GCTGCTGAAA |
| 1621 | GAAGCGGGTA TCGGGTTTGC GATGCCGGTT GCTCCGCTGT TTGAAACCCT CGATGATCTG |
| 1681 | AACAACGCCA ACGATGTCAT GACCCAGCTG CTCAATATTG ACTGGTATCG TGGCCTGATT |
| 1741 | CAGGGCAAAC AGATGGTGAT GATTGGCTAT TCCGACTCAG CAAAAGATGC GGGAGTGATG |
| 1801 | GCAGCTTCCT GGGCGCAATA TCAGGCACAG GATGCATTAA TCAAAACCTG CGAAAAAGCG |
| 1861 | GGTATTGAGC TGACGTTGTT CCACGGTCGC GGCGGTTCCA TTGGTCGCGG CGGCGCACCT |
| 1921 | GCTCATGCGG CGCTGCTGTC ACAACCGCCA GGAAGCCTGA AAGGCGGCCT GCGCGTAACC |
| 1981 | GAACAGGGCG AGATGATCCG CTTTAAATAT GGTCTGCCAG AAATCACCGT CAGCAGCCTG |
| 2041 | TCGCTTTATA CCGGGGCGAT TCTGGAAGCC AACCTGCTGC CACCGCCGGA GCCGAAAGAG |
| 2101 | AGCTGGCGTC GCATTATGGA TGAACTGTCA GTCATCTCCT GCGATGTCTA CCGCGGCTAC |
| 2161 | GTACGTGAAA ACAAAGATTT TGTGCCTTAC TTCCGCTCCG CTACGCCGGA ACAAGAACTG |
| 2221 | GGCAAACTGC CGTTGGGTTC ACGTCCGGCG AAACGTCGCC CAACCGGCGG CGTCGAGTCA |
| 2281 | CTACGCGCCA TTCCGTGGAT CTTCGCCTGG ACGCAAAACC GTCTGATGCT CCCCGCCTGG |
| 2341 | CTGGGTGCAG GTACGGCGCT GCAAAAAGTG GTCGAAGACG GCAAACAGAG CGAGCTGGAG |
| 2401 | GCTATGTGCC GCGATTGGCC ATTCTTCTCG ACGCGTCTCG GCATGCTGGA GATGGTCTTC |
| 2461 | GCCAAAGCAG ACCTGTGGCT GGCGGAATAC TATGACCAAC GCCTGGTAGA CAAAGCACTG |
| 2521 | TGGCCGTTAG GTAAAGAGTT ACGCAACCTG CAAGAAGAAG ACATCAAAGT GGTGCTGGCG |
| 2581 | ATTGCCAACG ATTCCCATCT GATGGCCGAT CTGCCGTGGA TTGCAGAGTC TATTCAGCTA |
| 2641 | CGGAATATTT ACACCGACCC GCTGAACGTA TTGCAGGCCG AGTTGCTGCA CCGCTCCCGC |
| 2701 | CAGGCAGAAA AAGAAGGCCA GGAACCGGAT CCTCGCGTCG AACAAGCGTT AATGGTCACT |
| 2761 | ATTGCCGGGA TTGCGGCAGG TATGCGTAAT ACCGGCTAAT CTTCCTCTTC TGCAAACCCT |
| 2821 | CGTGCTTTTG CGCGAGGGTT TTCTGAAATA CTTCTGTTCT AACACCCTCG TTT |

SEQ ID NO 5. rhtA and upstream and downstream regulatory regions
(rhtA translational start and stop codons are in bold)

| | |
|---|---|
| 1 | AATCCTGGCG CATTTTAGTC AAAACGGGGG AAAATTTTTT CAACAAATGC TCAACCAGCA |
| 61 | TTGGGTATAT CCAGTACACT CCACGCTTTA CTTAAGTCTA GATATTTGTG GGAGAAAGGA |
| 121 | TGCCTGGTTC ATTACGTAAA ATGCCGGTCT GGTTACCAAT AGTCATATTG CTCGTTGCCA |
| 181 | TGGCGTCTAT TCAGGGTGGA GCCTCGTTAG CTAAGTCACT TTTTCCTCTG GTGGGCGCAC |
| 241 | CGGGTGTCAC TGCGCTGCGT CTGGCATTAG GAACGCTGAT CCTCATCGCG TTCTTTAAGC |
| 301 | CATGGCGACT GCGCTTTGCC AAAGAGCAAC GGTTACCGCT GTTGTTTTAC GGCGTTTCGC |
| 361 | TGGGTGGGAT GAATTATCTT TTTTATCTTT CTATTCAGAC AGTACCGCTG GGTATTGCGG |
| 421 | TGGCGCTGGA GTTCACCGGA CCACTGGCGG TGGCGCTGTT CTCTTCTCGT CGCCCGGTAG |
| 481 | ATTTCGTCTG GGTTGTGCTG GCGGTTCTTG GTCTGTGGTT CCTGCTACCG CTGGGGCAAG |
| 541 | ACGTTTCCCA TGTCGATTTA ACCGGCTGTG CGCTGGCACT GGGGGCCGGG GCTTGTTGGG |
| 601 | CTATTTACAT TTTAAGTGGG CAACGCGCAG GAGCGGAACA TGGCCCTGCG ACGGTGGCAA |
| 661 | TTGGTTCGTT GATTGCAGCG TTAATTTTCG TGCCAATTGG AGCGCTTCAG GCTGGTGAAG |
| 721 | CACTCTGGCA CTGGTCGGTT ATTCCATTGG GTCTGGCTGT CGCTATTCTC TCGACCGCTC |
| 781 | TGCCTTATTC GCTGGAAATG ATTGCCCTCA CCCGTTTGCC AACACGGACA TTTGGTACGC |
| 841 | TGATGAGCAT GGAACCGGCG CTGGCTGCCG TTTCCGGGAT GATTTTCCTC GGAGAAACAC |
| 901 | TGACACCCAT ACAGCTACTG GCGCTCGGCG CTATCATCGC CGCTTCAATG GGGTCTACGC |
| 961 | TGACAGTACG CAAAGAGAGC AAAATAAAAG AATTAGACAT TAATTAAATT TACATTTCTG |
| 1021 | CATGGTTATG CATAACCATG CAGAATTTCT CGCTACTTTT CCTCTACACC GTCTTTATAT |
| 1081 | ATCGAATTAT GCAAAAGCAT ATTTATTCCG AAAATTCCTG GCGAGCAGAT AAATAAGAAT |
| 1141 | TGTTCTTATC AATATATCTA A |

SEQ ID NO 6. lacI coding region and upstream and downstream
regulatory regions. (lacI translational start and stop
codons are in bold)

| | |
|---|---|
| 1 | TCGGCGCAAA AAACATTATC CAGAACGGGA GTGCGCCTTG AGCGACACGA ATTATGCAGT |
| 61 | GATTTACGAC CTGCACAGCC ATACCACAGC TTCCGATGGC TGCCTGACGC CAGAAGCATT |
| 121 | GGTGCACCGT GCAGTCGATA AGCCCGGATC CTCTACGCCG GACGCATCGT GGCCGGCATC |
| 181 | ACCGGCGCCA CAGGTGCGGT TGCTGGCGCC TATATCGCCG ACATCACCGA TGGGGAAGAT |
| 241 | CGGGCTCGCC ACTTCGGGCT CATGAGCGCT TGTTTCGGCG TGGGTATGGT GGCAGGCCCC |
| 301 | GTGGCCGGGG GACTGTTGGG CGCCATCCTG CCTCGCGCGT TTCGGTGATG ACGGTGAAAA |
| 361 | CCTCTGACAC ATGCAGCTCC CGGAGACGGT CACAGCTTGT CTGTAAGCGG ATGCCGGGAG |
| 421 | CAGACAAGCC CGTCAGGGCG CGTCAGCGGG TGTTGGCGGG TGTCGGGGCG CAGCCATGAC |
| 481 | CCCCTCGACC TGCAGCAATT CCGACACCAT GGAATGGTGC AAAACCTTTC GCGGTATGGC |
| 541 | ATGATAGCGC CCGGAAGAGA GTCAATTCAG GGTGGTGAAT GTGAAACCAG TAACGTTATA |
| 601 | CGATGTCGCA GAGTATGCCG GTGTCTCTTA TCAGACCGTT TCCCGCGTGG TGAACCAGGC |
| 661 | CAGCCACGTT TCTGCGAAAA CGCGGGAAAA AGTGGAAGCG GCGATGGCGG AGCTGAATTA |
| 721 | CATTCCCAAC CGCGTGGCAC AACAACTGGC GGGCAAACAG TCGTTGCTGA TTGGCGTTGC |
| 781 | CACCTCCAGT CTGGCCCTGC ACGCGCCGTC GCAAATTGTC GCGGCGATTA AATCTCGCGC |
| 841 | CGATCAACTG GGTGCCAGCG TGGTGGTGTC GATGGTAGAA CGAAGCGGCG TCGAAGCCTG |
| 901 | TAAAGCGGCG GTGCACAATC TTCTCGCGCA ACGCGTCAGT GGGCTGATCA TTAACTATCC |
| 961 | GCTGGATGAC CAGGATGCCA TTGCTGTGGA AGCTGCCTGC ACTAATGTTC CGGCGTTATT |
| 1021 | TCTTGATGTC TCTGACCAGA CACCCATCAA CAGTATTATT TTCTCCCATG AAGACGGTAC |
| 1081 | GCGACTGGGC GTGGAGCATC TGGTCGCATT GGGTCACCAG CAAATCGCGC TGTTAGCGGG |
| 1141 | CCCATTAAGT TCTGTCTCGG CGCGTCTGCG TCTGGCTGGC TGGCATAAAT ATCTCACTCG |
| 1201 | CAATCAAATT CAGCCGATAG CGGAACGGGA AGGCGACTGG AGTGCCATGT CCGGTTTTCA |
| 1261 | ACAAACCATG CAAATGCTGA ATGAGGGCAT CGTTCCCACT GCGATGCTGG TTGCCAACGA |
| 1321 | TCAGATGGCG CTGGGCGCAA TGCGCGCCAT TACCGAGTCC GGGCTGCGCG TTGGTGCGGA |
| 1381 | TATCTCGGTA GTGGGATACG ACGATACCGA AGACAGCTCA TGTTATATCC CGCCGTTAAC |
| 1441 | CACCATCAAA CAGGATTTTC GCCTGCTGGG GCAAACCAGC GTGGACCGCT TGCTGCAACT |
| 1501 | CTCTCAGGGC CAGGCGGTGA AGGGCAATCA GCTGTTGCCC GTCTCACTGG TGAAAAGAAA |
| 1561 | AACCACCCTG GCGCCCAATA CGCAAACCGC CTCTCCCCGC GCGTTGGCCG ATTCATTAAT |
| 1621 | GCAGCTGGCA CGACAGGTTT CCCGACTGGA AAGCGGGCAG TGAGCGCAAC GCAATTAATG |
| 1681 | TAAGTTAGCT CACTCATTAG G |

SEQ ID NO 7. rhtB coding region

| | |
|---|---|
| 1 | atgaccttag aatggtggtt tgcctacctg ctgacatcga tcattttaag cctgtcgcca |
| 61 | ggctctggtg caatcaacac tatgaccacc tcgctcaacc acggttatcg cggcgcggtg |
| 121 | gcgtctattg ctgggcttca gaccggactg gcgattcata ttgtgctggt tggcgtgggg |
| 181 | ttggggacgc tattttcccg ctcagtgatt gcgtttgaag tgttgaagtg ggcaggcgcg |
| 241 | gcttacttga tttggctggg aatccagcag tggcgcgccg ctggtgcaat tgaccttaaa |
| 301 | tcgctggcct ctactcaatc gcgtcgacat ttgttccagc gcgcagtttt tgtgaatctc |
| 361 | accaatccca aaagtattgt gtttctggcg gcgctatttc cgcaattcat catgccgcaa |
| 421 | cagccgcaac tgatgcagta tatcgtgctc ggcgtcacca ctattgtggt cgatattatt |
| 481 | gtgatgatcg gttacgccac ccttgctcaa cggattgctc tatggattaa aggaccaaag |
| 541 | cagatgaagg cgctgaataa gattttcggc tcgttgttta tgctggtggg agcgctgtta |
| 601 | gcatcggcga ggcatgcgtg a |

What is claimed is:

1. A recombinant E. coli bacterium for producing L-homoserine, the recombinant bacterium comprising:
a. one or more exogenous nucleic acids encoding a polypeptide with aspartokinase activity,
b. one or more exogenous nucleic acids encoding a polypeptide with homoserine dehydrogenase activity,
c. one or more exogenous nucleic acids encoding a polypeptide with phosphoenolpyruvate carboxylase activity,
d. one or more exogenous nucleic acids encoding a polypeptide with homoserine transport activity, and
e. attenuated expression of the genomic nucleic acid encoding a polypeptide with homoserine kinase activity,
wherein one or more of the exogenous nucleic acids are operably linked to a native promoter.

2. A recombinant E. coli bacterium of claim 1, wherein the one or more exogenous nucleic acids are introduced into the bacterium on a vector.

3. A recombinant E. coli bacterium of claim 2, wherein the vector is selected from the group consisting of a viral vector, a cosmid, a phage and a plasmid.

4. A recombinant E. coli bacterium of claim 3, wherein the vector is a plasmid.

5. A recombinant E. coli bacterium of claim 4, wherein the vector is an intermediate copy number plasmid.

6. A recombinant E. coli bacterium of claim 5, wherein the vector is pBR322.

7. A recombinant E. coli bacterium of claim 1, wherein the aspartokinase activity and homoserine dehydrogenase activity are encoded by the E. coli thrA nucleic acid.

8. A recombinant E. coli acterium of claim 7, wherein the aspartokinase activity and homoserine dehydrogenase activity are encoded by a mutant thrA nucleic acid encoding a polypeptide resistant to feedback inhibition.

9. A recombinant E. coli bacterium of claim 1, wherein the phosphoenolpyruvate carboxylase activity is encoded by the E. coli ppc nucleic acid.

10. A recombinant E. coli bacterium of claim 1, wherein the homoserine transport activity is encoded by the E. coli rhtA nucleic acid.

11. A recombinant E. coli bacterium of claim 1, wherein the homoserine transport activity is encoded by the E. coli rhtA23 nucleic acid.

12. A recombinant E. coli bacterium of claim 1, wherein the homoserine transport activity is encoded by the E. coli rhtB nucleic acid.

13. A recombinant E. coli bacterium of claim 1, wherein the genomic nucleic acid encoding a polypeptide with homoserine kinase activity is encoded by the E. coli thrB nucleic acid.

14. A recombinant E. coli bacterium of claim 1, wherein the expression of the genomic nucleic acid encoding a polypeptide with homoserine kinase activity is attenuated by deleting part or all of the genomic nucleic acid encoding a polypeptide with homoserine kinase activity.

15. A recombinant E. coli bacterium of claim 1, wherein the one or more exogenous nucleic acids encoding polypeptides with aspartokinase activity, homoserine dehydrogenase activity, phosphoenolpyruvate carboxylase activity, and homoserine transport activity are operably linked to a native promoter.

16. A recombinant E. coli bacterium of claim 1, wherein the one or more exogenous nucleic acids encoding one or more polypeptides with phosphoenolpyruvate carboxylase activity, and homoserine transport activity are operably linked to a native promoter.

17. A recombinant E. coli bacterium of claim 1, wherein the one or more exogenous nucleic acids encoding one or more polypeptides with aspartokinase activity, homoserine dehydrogenase activity, and homoserine transport activity are operably linked to a native promoter.

18. A recombinant E. coli bacterium of claim 1, wherein the bacterium comprises
a. an exogenous E. coli thrA nucleic acid,
b. an exogenous E. coli ppc nucleic acid,
c. at least one exogenous sequence of the group consisting of an E. coli rhtA nucleic acid, an E. coli rhtA23 nucleic acid, and an E. coli rhtB nucleic acid, and
d. attenuated expression of the genomic E. coli thrB nucleic acid,
wherein one or more of the exogenous nucleic acids are operably linked to a native promoter.

19. A recombinant E. coli bacterium of claim 1, wherein the bacterium comprises
a. an exogenous E. coli thrA nucleic acid operably linked to a native E. coli thrA promoter,
b. an exogenous E. coli ppc nucleic acid operably linked to a native E. coli ppc promoter,
c. at least one exogenous sequence of the group consisting of an E. coli rhtA nucleic acid operably linked to a native E. coli rhtA promoter, an E. coli rhtA23 nucleic acid operably linked to a native E. coli rhtA promoter, and an E. coli rhtB nucleic acid operably linked to a native E. coli rhtB promoter, and
d. attenuated expression of the genomic E. coli thrB nucleic acid.

20. A method of producing L-homoserine the method comprising, cultivating a recombinant bacterium described in claim 1 in a culture medium and collecting the L-homoserine from the medium.


SEQUENCE LISTING (7 sequences)

SEQ ID NO: 1 - DNA, 336 bp, Escherichia coli
agcttttcattctgactgcaacgggcaatatgtctctgtgtggattaaaaaaagagtgtctgatagcagcttctgaactggttacctgccgtgagtaaattaaaattttattgacttaggtcactaaatactttaaccaatataggcatagcgcacagacagataaaaattacagagtacacaacatccatgaaacgcattagcaccaccattaccaccaccatcaccattaccacaggtaacggtgcgggctgacgcgtacaggaaacacagaaaaaagcccgcacctgacagtgcgggctttttttttcgaccaaaggtaacgaggtaacaacc

SEQ ID NO: 2 - DNA, 2463 bp, Escherichia coli
atgcgagtgttgaagttcggcggtacatcagtggcaaatgcagaacgttttctgcgtgttgccgatattctggaaagcaatgccaggcaggggcaggtggccaccgtcctctctgcccccgccaaaatcaccaaccacctggtggcgatgattgaaaaaaccattagcggccaggatgctttacccaatatcagcgatgccgaacgtatttttgccgaacttttgacgggactcgccgccgcccagccggggttcccgctggcgcaattgaaaactttcgtcgatcaggaatttgcccaaataaaacatgtcctgcatggcattagtttgttggggcagtgcccggatagcatcaacgctgcgctgatttgccgtggcgagaaaatgtcgatcgccattatggccggcgtattagaagcgcgcggtcacaacgttactgttatcgatccggtcgaaaaactgctggcagtggggcattacctcgaatctaccgtcgatattgctgagtccacccgccgtattgcggcaagccgcattccggctgatcacatggtgctgatggcaggtttcaccgccggtaatgaaaaaggcgaactggtggtgcttggacgcaacggttccgactactctgctgcggtgctggctgcctgtttacgcgccgattgttgcgagatttggacggacgttgacggggtctatacctgcgacccgcgtcaggtgcccgatgcgaggttgttgaagtcgatgtcctaccaggaagcgatggagctttcctacttcggcgctaaagttcttcacccccgcaccattacccccatcgcccagttccagatcccttgcctgattaaaaataccggaaatcctcaagcaccaggtacgctcattggtgccagccgtgatgaagacgaattaccggtcaagggcatttccaatctgaataacatggcaatgttcagcgtttctggtccggggatgaaagggatggtcggcatggcggcgcgcgtctttgcagcgatgtcacgcgcccgtatttccgtggtgctgattacgcaatcatcttccgaatacagcatcagtttctgcgttccacaaagcgactgtgtgcgagctgaacgggcaatgcaggaagagttctacctggaactgaaagaaggcttactggagccgctggcagtgacggaacggctggccattatctcggtggtaggtgatggtatgcgcaccttgcgtgggatctcggcgaaattctttgccgcactggcccgcgccaatatcaacattgtcgccattgctcagggatcttctgaacgctcaatctctgtcgtggtaaataacgatgatgcgaccactggcgtgcgcgttactcatcagatgctgttcaataccgatcaggttatcgaagtgtttgtgattggcgtcggtggcgttggcggtgcgctgctggagcaactgaagcgtcagcaaagctggctgaagaataaacatatcgacttacgtgtctgcggtgttgccaactcgaaggctctgctcaccaatgtacatggccttaatctggaaaactggcaggaagaactggcgcaagccaaagagccgtttaatctcgggcgcttaattcgcctcgtgaaagaatatcatctgctgaacccggtcattgttgactgcacttccagccaggcagtggcggatcaatatgccgacttcctgcgcgaaggtttccacgttgtcacgccgaacaaaaaggccaacacctcgtcgatggattactaccatcagttgcgttatgcggcggaaaaatcgcggcgtaaattcctctatgacaccaacgttggggctggattaccggttattgagaacctgcaaaatctgctcaatgcaggtgatgaattgatgaagttctccggcattctttctggttcgctttcttatatcttcggcaagttagacgaaggcatgagtttctccgaggcgaccacgctggcgcgggaaatgggttataccgaaccggacccgcgagatgatctttctggtatggatgtggcgcgtaaactattgattctcgctcgtgaaacgggacgtgaactggagctggcggatattgaaattgaacctgtgctgcccgcagagtttaacgccgagggtgatgttgccgcttttatggcgaatctgtcacaactcgacgatctctttgccgcgcgcgtggcgaaggcccgtgatgaaggaaaagttttgcgctatgttggcaatattgatgaagatggcgtctgccgcgtgaagattgccgaagtggatggtaatgatccgctgttcaaagtgaaaaatggcgaaaacgccctggccttctatagccactattatcagccgctgccgttggtactgcgcggatatggtgcgggcaatgacgttacagctgccggtgtctttgctgatctgctacgtaccctctcatggaagttaggagtctga

SEQ ID NO: 3 - DNA, 665 bp, Escherichia coli
aatctattcattatctcaatcaggccgggtttgcttttatgcagcccggcttttttatgaagaaattatggagaaaaatgacagggaaaaaggagaaattctcaataaatgcggtaacttagagattaggattgcggagaataacaaccgccgttctcatcgagtaatctccggatatcgacccataacgggcaatgataaaaggagtaacctgtgaaaaagatgcaatctatcgtactcgcactttccctggttctggtcgctcccatggcagcacaggctgcggaaattacgttagtcccgtcagtaaaattacagataggcgatcgtgataatcgtggctattactgggatggaggtcactggcgcgaccacggctggtggaaacaacattatgaatggcgaggcaatcgctggcacctacacggaccgccgccaccgccgcgccaccataagaaagctcctcatgatcatcacggcggtcatggtccaggcaaacatcaccgctaaatgacaaatgccgggtaacaatccggcattcagcgcctgatgcgacgctggcgcgtcttatcaggcctacgttaattctgcaatatattgaatctgcatgcttttgtaggcaggataaggcgttcacgccgcatccggcattgactgcaaactta

SEQ ID NO: 4 - DNA, 2873 bp, Escherichia coli
tgctgaagcgatttcgcagcatttgacgtcaccgcttttacgtggctttataaaagacgacgaaaagcaaagcccgagcatattcgcgccaatgcgacgtgaaggatacagggctatcaaacgataagatggggtgtctggggtaatatgaacgaacaatattccgcattgcgtagtaatgtcagtatgctcggcaaagtgctgggagaaaccatcaaggatgcgttgggagaacacattcttgaacgcgtagaaactatccgtaagttgtcgaaatcttcacgcgctggcaatgatgctaaccgccaggagttgctcaccaccttacaaaatttgtcgaacgacgagctgctgcccgttgcgcgtgcgtttagtcagttcctgaacctggccaacaccgccgagcaataccacagcatttcgccgaaaggcgaagctgccagcaacccggaagtgatcgcccgcaccctgcgtaaactgaaaaaccagccggaactgagcgaagacaccatcaaaaaagcagtggaatcgctgtcgctggaactggtcctcacggctcacccaaccgaaattacccgtcgtacactgatccacaaaatggtggaagtgaacgcctgtttaaaacagctcgataacaaagatatcgctgactacgaacacaaccagctgatgcgtcgcctgcgccagttgatcgcccagtcatggcataccgatgaaatccgtaagctgcgtccaagcccggtagatgaagccaaatggggctttgccgtagtggaaaacagcctgtggcaaggcgtaccaaattacctgcgcgaactgaacgaacaactggaagagaacctcggctacaaactgcccgtcgaatttgttccggtccgttttacttcgtggatgggcggcgaccgcgacggcaacccgaacgtcactgccgatatcacccgccacgtcctgctactcagccgctggaaagccaccgatttgttcctgaaagatattcaggtgctggtttctgaactgtcgatggttgaagcgacccctgaactgctggcgctggttggcgaagaaggtgccgcagaaccgtatcgctatctgatgaaaaacctgcgttctcgcctgatggcgacacaggcatggctggaagcgcgcctgaaaggcgaagaactgccaaaaccagaaggcctgctgacacaaaacgaagaactgtgggaaccgctctacgcttgctaccagtcacttcaggcgtgtggcatgggtattatcgccaacggcgatctgctcgacaccctgcgccgcgtgaaatgtttcggcgtaccgctggtccgtattgatatccgtcaggagagcacgcgtcataccgaagcgctgggcgagctgacccgctacctcggtatcggcgactacgaaagctggtcagaggccgacaaacaggcgttcctgatccgcgaactgaactccaaacgtccgcttctgccgcgcaactggcaaccaagcgccgaaacgcgcgaagtgctcgatacctgccaggtgattgccgaagcaccgcaaggctccattgccgcctacgtgatctcgatggcgaaaacgccgtccgacgtactggctgtccacctgctgctgaaagaagcgggtatcgggtttgcgatgccggttgctccgctgtttgaaaccctcgatgatctgaacaacgccaacgatgtcatgacccagctgctcaatattgactggtatcgtggcctgattcagggcaaacagatggtgatgattggctattccgactcagcaaaagatgcgggagtgatggcagcttcctgggcgcaatatcaggcacaggatgcattaatcaaaacctgcgaaaaagcgggtattgagctgacgttgttccacggtcgcggcggttccattggtcgcggcggcgcacctgctcatgcggcgctgctgtcacaaccgccaggaagcctgaaaggcggcctgcgcgtaaccgaacagggcgagatgatccgctttaaatatggtctgccagaaatcaccgtcagcagcctgtcgctttataccggggcgattctggaagccaacctgctgccaccgccggagccgaaagagagctggcgtcgcattatggatgaactgtcagtcatctcctgcgatgtctaccgcggctacgtacgtgaaaacaaagattttgtgccttacttccgctccgctacgccggaacaagaactgggcaaactgccgttgggttcacgtccggcgaaacgtcgcccaaccggcggcgtcgagtcactacgcgccattccgtggatcttcgcctggacgcaaaaccgtctgatgctccccgcctggctgggtgcaggtacggcgctgcaaaaagtggtcgaagacggcaaacagagcgagctggaggctatgtgccgcgattggccattcttctcgacgcgtctcggcatgctggagatggtcttcgccaaagcagacctgtggctggcggaatactatgaccaacgcctggtagacaaagcactgtggccgttaggtaaagagttacgcaacctgcaagaagaagacatcaaagtggtgctggcgattgccaacgattcccatctgatggccgatctgccgtggattgcagagtctattcagctacggaatatttacaccgacccgctgaacgtattgcaggccgagttgctgcaccgctcccgccaggcagaaaaagaaggccaggaaccggatcctcgcgtcgaacaagcgttaatggtcactattgccgggattgcggcaggtatgcgtaataccggctaatcttcctcttctgcaaaccctcgtgcttttgcgcgagggttttctgaaatacttctgttctaacaccctcgttt

SEQ ID NO: 5 - DNA, 1161 bp, Escherichia coli
aatcctggcgcattttagtcaaaacgggggaaaattttttcaacaaatgctcaaccagcattgggtatatccagtacactccacgctttacttaagtctagatatttgtgggagaaaggatgcctggttcattacgtaaaatgccggtctggttaccaatagtcatattgctcgttgccatggcgtctattcagggtggagcctcgttagctaagtcactttttcctctggtgggcgcaccgggtgtcactgcgctgcgtctggcattaggaacgctgatcctcatcgcgttctttaagccatggcgactgcgctttgccaaagagcaacggttaccgctgttgttttacggcgtttcgctgggtgggatgaattatcttttttatctttctattcagacagtaccgctgggtattgcggtggcgctggagttcaccggaccactggcggtggcgctgttctcttctcgtcgcccggtagatttcgtctgggttgtgctggcggttcttggtctgtggttcctgctaccgctggggcaagacgtttcccatgtcgatttaaccggctgtgcgctggcactgggggccggggcttgttgggctatttacattttaagtgggcaacgcgcaggagcggaacatggccctgcgacggtggcaattggttcgttgattgcagcgttaattttcgtgccaattggagcgcttcaggctggtgaagcactctggcactggtcggttattccattgggtctggctgtcgctattctctcgaccgctctgccttattcgctggaaatgattgccctcacccgtttgccaacacggacatttggtacgctgatgagcatggaaccggcgctggctgccgtttccgggatgattttcctcggagaaacactgacacccatacagctactggcgctcggcgctatcatcgccgcttcaatggggtctacgctgacagtacgcaaagagagcaaaataaaagaattagacattaattaaatttacatttctgcatggttatgcataaccatgcagaatttctcgctacttttcctctacaccgtctttatatatcgaattatgcaaaagcatatttattccgaaaattcctggcgagcagataaataagaattgttcttatcaatatatctaa

SEQ ID NO: 6 - DNA, 1701 bp, Escherichia coli
tcggcgcaaaaaacattatccagaacgggagtgcgccttgagcgacacgaattatgcagtgatttacgacctgcacagccataccacagcttccgatggctgcctgacgccagaagcattggtgcaccgtgcagtcgataagcccggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatcctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaccccctcgacctgcagcaattccgacaccatggaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattagg

SEQ ID NO: 7 - DNA, 621 bp, Escherichia coli
atgaccttagaatggtggtttgcctacctgctgacatcgatcattttaagcctgtcgccaggctctggtgcaatcaacactatgaccacctcgctcaaccacggttatcgcggcgcggtggcgtctattgctgggcttcagaccggactggcgattcatattgtgctggttggcgtggggttggggacgctattttcccgctcagtgattgcgtttgaagtgttgaagtgggcaggcgcggcttacttgatttggctgggaatccagcagtggcgcgccgctggtgcaattgaccttaaatcgctggcctctactcaatcgcgtcgacatttgttccagcgcgcagtttttgtgaatctcaccaatcccaaaagtattgtgtttctggcggcgctatttccgcaattcatcatgccgcaacagccgcaactgatgcagtatatcgtgctcggcgtcaccactattgtggtcgatattattgtgatgatcggttacgccacccttgctcaacggattgctctatggattaaaggaccaaagcagatgaaggcgctgaataagattttcggctcgttgtttatgctggtgggagcgctgttagcatcggcgaggcatgcgtga